(19) United States
(12) Patent Application Publication
(10) Pub. No.: US 2006/0173350 A1
(43) Pub. Date: Aug. 3, 2006

(54) Systems and methods for three dimensional imaging with an orientation adjustable array

(75) Inventors: Jian R. Yuan, Hayward, CA (US); Pei Jei Cao, Fremont, CA (US); Richard Romley, Tracy, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(73) Assignee: SciMed Life Systems, Inc.

(21) Appl. No.: 11/034,339

(22) Filed: Jan. 11, 2005

(51) Int. Cl. A61B 8/14

(52) U.S. Cl. 600/466; 600/459

(57) ABSTRACT

The systems and methods described herein allow for three dimensional imaging with a medical ultrasound imaging system having an orientation adjustable imaging device. The imaging device can include a transducer array configured to image an imaging field in two dimensions. The imaging device can also include an orientation adjustment unit configured to adjust the orientation of the array in a third dimension. The array can be configured to image the two dimensional imaging field at multiple different orientations. An image processing system can be communicatively coupled with the array and configured to assemble the image data collected across each imaging field at multiple orientations of the array. The assembled data can then be displayed as a three dimensional image.

### FIELD OF THE INVENTION

The systems and methods relate generally to medical ultrasound imaging, and more particularly to three dimensional ultrasound imaging with an orientation adjustable array.

### BACKGROUND INFORMATION

The ability to perform three-dimensional (3D) ultrasound imaging of the interior of a living being provides numerous diagnostic and therapeutic advantages. However, 3D imaging with intravascular or other internally inserted imaging systems, such as intravascular ultrasound or intracardiac echocardiography (ICE) imaging systems, is difficult. This is mainly because of the size constraints inherent in the use of internal imaging devices.

For instance, conventional 3D imaging systems require a two-dimensional (2D) phased array having numerous transducer elements. This 2D array provides a steerable imaging beam which images in one direction and can be steered in two additional directions, thus providing 3D capability. However, 2D arrays are very costly and typically too large for insertion into most regions of a living being, such as narrow blood vessels. Furthermore, each element is typically coupled with a separate communication line, e.g., a cable, in order to communicate with an external imaging system. These communication lines add undesirable cross-sectional area to the insertable device (such as a catheter) being used to deploy and navigate the array within the body. This added cross-sectional area, or width, can also prevent use of the array within narrow regions of the body. Finally, 2D arrays are susceptible to cross-talk between elements, which can significantly degrade performance.

Other conventional 3D imaging systems use a single element transducer mounted on the distal end of a rotating drive shaft. This single element transducer images one dimensionally in a radial direction perpendicular or transverse to the central axis of the drive shaft. When the transducer is rotated in a second direction, the image data collected can be used to generate a 2D cross-sectional image of the body tissue. The driveshaft is typically located within an outer sheath and can be slid proximally and distally within the sheath along the central axis of the drive shaft. Multiple 2D cross-sectional images can be obtained at different positions along the central axis. An image processing system can then be used to assemble, or reconstruct these images into a 3D image of the body tissue. However, this process cannot be performed in real-time since it requires the reconstruction of previously obtained 2D images.

Accordingly, there is a need for improved systems and methods for 3D imaging which overcome the shortcomings of conventional 3D imaging systems.

### SUMMARY

The systems and methods described herein provide for a medical ultrasound imaging system configured for 3D imaging of a living being with an orientation adjustable imaging device insertable into a living being and configured to image the interior of the living being. In one example embodiment as described below, the imaging device includes an ultrasound array having an imaging field and an orientation adjustment unit coupled with the array and configured to adjust the orientation of the array. The array can include multiple transducer elements configured as a linear array arranged along a one dimensional axis. The array can preferably image a two-dimensional imaging field such that when the orientation of the array is adjusted in a third dimension, image data from a three-dimensional region can be collected.

The orientation adjustment unit can be configured to adjust the orientation of the array in any manner. In one embodiment, orientation adjustment unit adjusts the pitch of the array about an axis. The orientation adjustment unit can include an orientation control unit configured to control the orientation of the array, control the rate of adjustment of the array and optionally determine the orientation of the array. The orientation control unit can control the orientation of the array in any manner, such as electrically, mechanically, magnetically and the like. The orientation adjustment unit can also include an adjustable mounting for mounting the array thereon. In one embodiment, the adjustable mounting is a flexible circuit having a multiplexer for multiplexing signals communicated to and from the array.

The imaging system can also include an image processing system communicatively coupled with the array. In an example embodiment as described below, the image processing system can be configured to control the imaging direction of the array and can be configured to, receive an output signal from each element in the array, where one or more of the output signals are representative of an echo received in the imaging direction. This image processing system can also be configured to process the received output signals and generate a three-dimensional image therefrom. In one example embodiment, the image processing system can be configured to process the one or more output signals into echo data and store the echo data in an echogenic record, where one echogenic record is generated for each imaging direction imaged by the array. The image processing system can be configured to store the echogenic records generated at each orientation of the array as a separate image data set and can also be configured to generate a three-dimensional image from the image data sets corresponding to multiple orientations of the array.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims. It is also intended that the invention is not limited to require the details of the example embodiments.

### BRIEF DESCRIPTION OF THE FIGURES

The details of the invention, including fabrication, structure and operation, may be gleaned in part by study of the accompanying figures, in which like reference numerals refer to like segments.

FIGS. 1A-C are block diagrams depicting an example embodiment of an medical imaging system with an orientation adjustable imaging device.

FIG. 2A is a perspective view depicting an example embodiment of an orientation adjustable imaging device.

FIGS. 2B-C are top down views depicting additional example embodiments of an orientation adjustable imaging device.

FIG. 3 is a block diagram depicting another example embodiment of a medical imaging system with an orientation adjustable imaging device.

FIG. 4 is a schematic view depicting an example embodiment of an orientation adjustable imaging device.

FIG. 5 is a block diagram depicting another example embodiment of a medical imaging system with a multiplexer.

FIG. 6 is a perspective view depicting another example embodiment of a medical imaging system with an orientation adjustable imaging device.

FIG. 7 is a block diagram depicting another example embodiment of a medical imaging system with an orientation adjustable imaging device.

### DETAILED DESCRIPTION

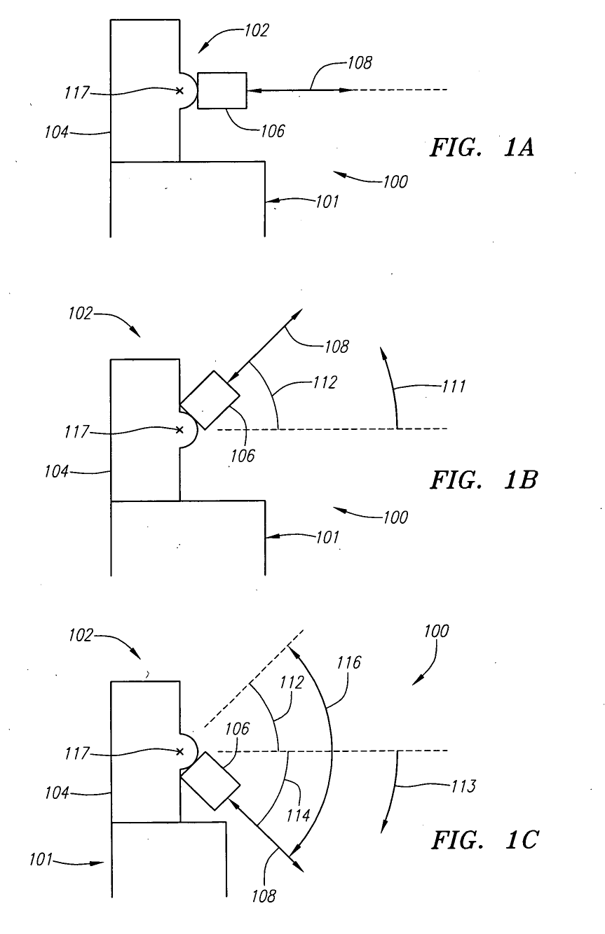

The systems and methods described herein provide for 3D imaging with a medical ultrasound imaging system using an orientation adjustable imaging device. FIGS. 1A-C depict one example embodiment of an ultrasound imaging system 100 having an orientation adjustable imaging device 102. Imaging device 102 is preferably a component of a flexible elongate medical device 101, such as a catheter, endoscope and the like, which is insertable into a living being and configured to allow imaging of the interior of the living being with imaging device 102. Imaging system 100 can be any type of ultrasound imaging system having an insertable imaging device 102, such as an IVUS imaging system, an ICE imaging system or other imaging systems. Imaging device 102 preferably includes an orientation adjustment unit 104 and an ultrasound transducer device 106 configured to image an imaging field 108, which is preferably 2D. Ultrasound transducer device 106 is preferably a transducer array, but can also be multiple transducer elements in a non-array configuration or a single element transducer. Orientation adjustment unit 104 is preferably configured to adjust the orientation of array 106 in a third dimension, indicated by directions 111 and 113, to allow array 106 to image a 3D region of the body.

In the embodiments depicted in FIGS. 1A-C, array 106 is adjustable over a range of motion 116. In this embodiment, array 106 is rotatable about axis 117. FIGS. 1A-C each depict array 106 at a separate orientation with motion range 116. FIG. 1A depicts array 106 positioned at a first orientation located approximately in the center of motion range 116. FIG. 1B depicts array 106 positioned at a second orientation where the pitch of array 106 has been adjusted in direction 111 by an angle 112, while FIG. 1C depicts array 106 positioned at a third orientation where the pitch of array 106 has been adjusted in direction 113 by an angle 114. Here, motion range 116 is approximately 120 degrees; however, the limits of motion range 116 are entirely dependent upon the needs of the application and can be set to any appropriate range or ranges.

At each orientation within range 116, array 106 can be used to image field 108. Preferably, array 106 sweeps back and forth across motion range 116 while at the same time collecting image data across 2D imaging field 108 that can be used to generate a 3D image. It should be noted that motion range 116 is not limited to motion only in directions 111 and 113. The orientation of array 106 can be adjusted in any manner and through any range of motion. For instance, motion range 116 can include up/down movement, left/right movement, forward/backward movement, rotational movement, tilting movement, pivoting movement, wobbling movement, oscillating movement and other types of movement.

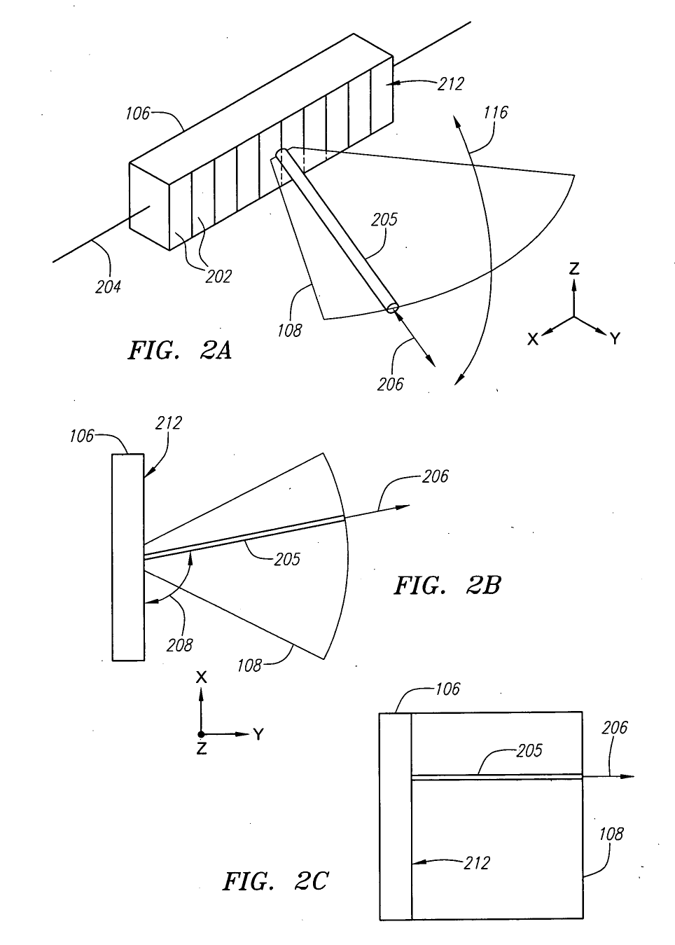

FIG. 2A depicts a perspective view of one example embodiment of array 106 configured as a linear, curved linear or one-dimensional (1D) phased array including a series of individual transducer elements 202 arranged along a common axis 204. In this embodiment, array 106 is configured to generate an imaging beam 205 in a variable direction 206. Specifically, array 106 can be configured to transmit an ultrasound signal beam 205 along direction 206 and receive echoes propagating back towards array 102 along direction 206, the echoes generally resulting from the collision of the transmitted ultrasound signal with body tissue. Direction 206 is variable, or steerable, and array 106 is preferably configured to image the body tissue in multiple different directions 206. In other embodiments of imaging device 102 that image only in one dimension, such as a single element transducer, orientation adjustment unit 104 is preferably configured to move the imaging device in two dimensions to allow for 3D imaging.

FIG. 2B depicts a top down view of an example embodiment of array 106 with a steerable imaging beam 205. Imaging beam 205 can be generated in multiple different directions 206, each at a different angular location 208 with respect to array 106. Here, the ultrasound beams 205 generated at each angular location 208 define the imaging field 108 of the array 106. Preferably, during an imaging procedure, the beam 205 images in direction 206 at one angular location 208 and then is adjusted, or steered, to a second adjacent angular location 208 and images again. In this manner, beam 205 can be swept across imaging field 108. Because imaging field 108 extends substantially in two directions, X and Y, the data collected from each sweep of imaging field 108 can be used to collect 2D image data of the body tissue.

In practice, beam 205 will have a finite cross-sectional area and imaging field 108 will extend into the Z direction by a small amount. However, this amount is generally negligible for 3D imaging purposes, so imaging field 108 is referred to herein as being substantially 2D. One of skill in the art will readily recognize that the shape of beam 205 can be adjusted to provide greater resolution in the Z direction as required by the needs of the application.

FIG. 2C depicts a top down view of another example embodiment of array 106. Here, array 106 is configured to image in multiple directions 206, each direction 206 being substantially perpendicular to the face 212 of array 106 and located at a different position along the face 212. By adjusting the position along face 212, beam 205 can be swept across imaging field 108 to collect 2D image data of the body tissue.

After collecting 2D image data over the imaging field 108 at a first orientation of array 106, the orientation adjustment unit 104 preferably adjusts the array 106 to a second orientation to collect 2D image data over the imaging field 108 at that orientation. This process repeats until 2D image data has been collected for a desired number of different orientations of array 106. This collected 2D image data can then be assembled, or reconstructed, by an image processing system 306 (described below) to generate a 3D image of the body tissue. Thus, in this embodiment a 1D array 106 can be used to generate a 3D image with superior quality than conventional systems, due in part to the reduced potential for cross-talk resulting from the use of a 1D array 106.

However, any type of transducer array 106 can be used including 2D arrays and other appropriate transducer configurations. Array 106 can be a linear or phased array. Array 106 can also be fabricated in any manner desired. For instance, array 106 can include piezoelectric transducer elements, micromachined ultrasound transducer (MUT) elements such as capacitive micromachined ultrasound transducers (CMUTs) or piezoelectric micromachined ultrasound transducers (PMUTs), or other known transducer array structures.

The rate at which the orientation of imaging device 102 is adjusted is dependent upon the needs of the application and can be as rapid or as slow as desired. Also, the orientation adjustment can be continuous or can proceed in a stepped fashion. The adjustment rate can also be related to the imaging frame rate of imaging system 100, for instance, to allow for real-time 3D imaging. In one example, a video frame may include image data collected from 100 separate imaging fields 108, each located at a different pitch within motion range 116. If the imaging frame rate is 30 frames per second, then each sweep of array 106 across motion range 116 can take no longer than 0.0333 seconds. If the pitch is adjusted in a stepped fashion and it takes 20 microseconds to image one imaging field 108, then the time to adjust array 106 from one pitch to the next can be no longer than 133 microseconds. It should be noted that these values serve only as an example and in no way limit the systems and methods described herein.

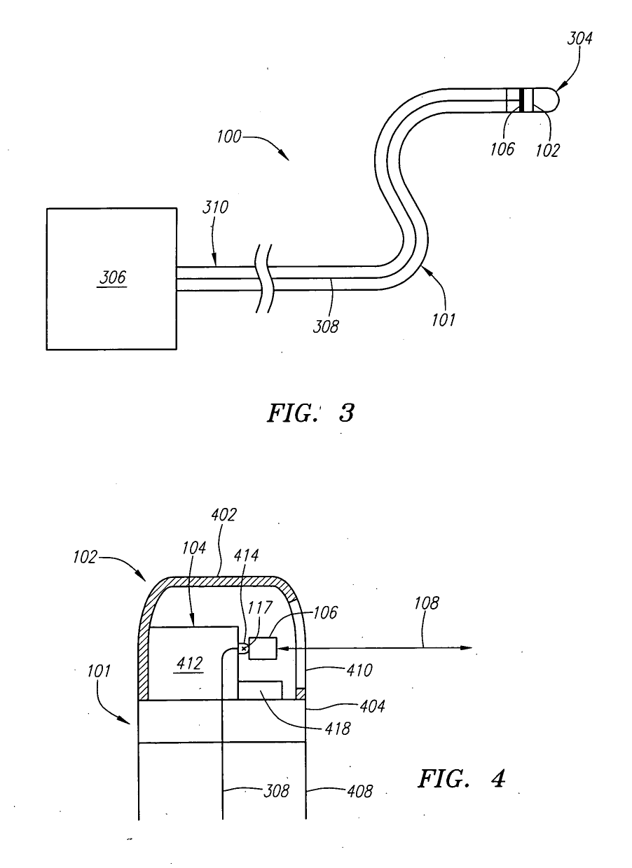

FIG. 3 depicts a block diagram of another example embodiment of imaging system 100. Here, array 106 is located at or near the distal end 304 of medical device 101 and is communicatively coupled with the image processing system 306 via one or more communication lines 308. Image processing system 306 is preferably located externally to the living being at the proximal end 310 of medical device 101. Image processing system 306 is preferably configured to control the imaging direction 206 of beam 205. Image processing system 306 is also preferably configured to receive an output signal from each element 202 in array 206 and process the output signal into echo data representative of an echo received by array 106 in direction 206.

In one embodiment, image processing system 306 is configured to store the echo data in an echogenic record, where each echogenic record includes the echo data received in direction 206 at one angular location 208 in the imaging field 108. One echogenic record can be generated for each angular location 208 in an imaging field 108 for one orientation of array 106. All of the echogenic records from a given imaging field 108 can then be grouped together by image processing system 306 into an image data set. Image processing system 306 is preferably configured to assemble each of the image data sets and generate a 3D image of the body tissue. Image processing system 306 preferably includes the processing hardware and/or software to generate the 3D images in real-time, or near real-time, for the benefit of the physician or technician operating system 100.

FIG. 4 depicts a schematic view of another example embodiment of imaging system 100 showing imaging device 102 in closer detail. Here, imaging device 102 includes a housing 402 coupled with a base structure 404. Base structure 404, in turn, is coupled to the distal end 406 of an elongate shaft 408. Elongate shaft 408 can be used to position imaging device 102 into proximity with the desired region for imaging, by moving the imaging device 102 along its longitudinal axis, for example. Array 106 and orientation adjustment unit 104 are preferably housed within a housing 402. Housing 402 can optionally include an imaging window 410 composed of a material that does not substantially interfere with the transmission or reception of the ultrasound signals, including known sonulucent materials. Window 410 can also be an aperture in housing 402. Preferably, window 410 is large enough to accommodate imaging across the entire motion range 116 of array.106. In another embodiment, an elongate tubular outer sheath (not shown) having an inner lumen is provided. The inner lumen can be configured to slidably receive imaging device 102 and shaft 408.

The term “orientation” is defined herein as the position of array 106 with respect to the structure or device used to move, navigate or guide array 106 within the living being. In this embodiment, although shaft 408 can be used to move the imaging device 102 within the living being, for instance to position imaging device 102 in proximity with the desired region for imaging, the orientation of array 106 remains adjustable even when shaft 408 is stationary.

In this embodiment, orientation adjustment unit 104 is configured to control the orientation of the array 106 and determine the orientation of array 106 at any given time, for instance, in order to allow tracking of array 106. Orientation adjustment unit 104 can include an orientation control unit 412 for controlling and determining the orientation of array 106. Orientation control unit 412 can be configured in any manner in accordance with the needs of the application.

For instance, orientation control unit 412 can be configured to electrically, mechanically or magnetically operate or control the orientation of array 106, or any combination thereof. In one example embodiment, orientation control unit 412 includes one or more actuators for adjusting the orientation of array 106. One example actuator that can be used is a piezo-film actuator, although the systems and methods described herein are not limited to such. In another embodiment, orientation control unit 412 includes a piezoelectric drive for orientation control of array 106. In yet another embodiment, orientation control unit 412 includes a rolling wheel and an electrical servo motor for powering the wheel, which is in turn coupled with array 106 by a wire or tether. Adjustment of the rolling wheel applies tension to the array via the wire or tether and can be used to control and adjust the orientation of array 106. Orientation adjustment unit 104 can also optionally include one or more sensors 418 for determining the orientation of array 106 at any given time. Sensors 418 can use any type of sensing technique such as electrical, optical, magnetic, capacitive, inductive etc.

Orientation control unit 412 can be adjustably coupled with array 106. For instance in one embodiment, orientation control unit 412 is a flexible circuit physically coupled with array 106. Alternatively, orientation adjustment unit 104 can also include a position adjustable mounting 414 for adjustably coupling array 106 with orientation control unit 412. Any type of position adjustable mounting 414 can be used in accordance with the needs of the application. For instance, in one embodiment, communication lines 308 are flexible and function as position adjustable mounting 414. In another embodiment, position adjustable mounting 414 is a hinge-type structure configured to limit the motion of array 106 to movement solely within motion range 116. It should be understood that these embodiments are only examples and in no way limit the systems and methods described herein.

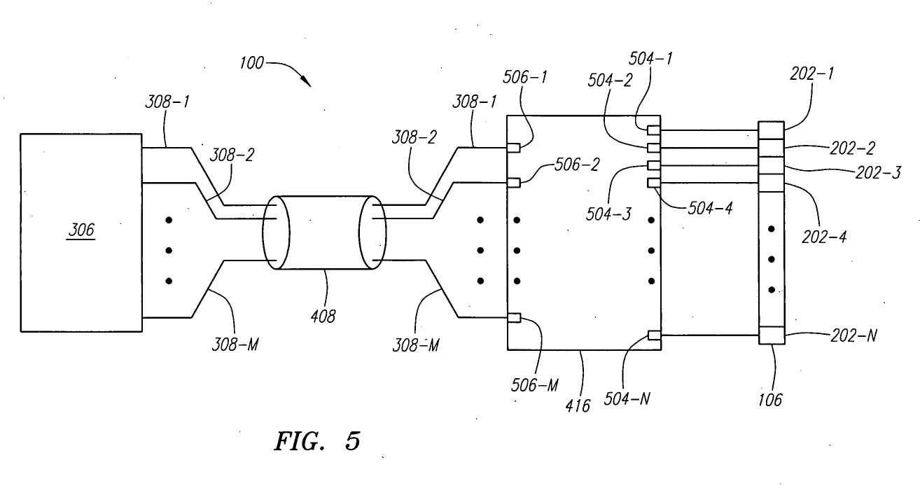

Orientation adjustment unit 104 can also include a multiplexer 416. FIG. 5 is a block diagram depicting an example embodiment of imaging device 102 with multiplexer 416. In this embodiment, each array element 202-1 through 202-N (where ‘N’ indicates that any number of elements 202 can be present) is coupled with a separate communication line 502-1 through 502-N. Multiplexer 416 includes communication ports 504-1 through 504-N coupled with each element 202-1 through 202-N by way of communication lines 502-1 through 502-N.

Multiplexer 416 also includes communication ports 506-1 through 506-M (where “M” indicates that any number of ports 506 can be present, unless otherwise noted). Each communication port 506-1 through 506-M is preferably coupled with a communication line 308-1 through 308-M and routed to image processing system 306 with shaft 408. Preferably, multiplexer 416 is an N:M multiplexer configured to multiplex the signals input to ports 504-1 through 504-N and output the multiplexed signals from ports 506-1 through 506-M, where M is less then N. Multiplexer 416 also preferably includes corresponding M:N demultiplexer circuitry to demultiplex the signals input to ports 506-1 through 506-M and output the demultiplexed signals from ports 504-1 through 504-N to array 106. Also, image processing system 306 preferably includes complementary multiplexing and demultiplexing hardware and/or software for communication with array 106.

The use of a multiplexer 416, with the value of M less than N, reduces the number of communication lines 308 necessary to transmit signals between array 106 and image processing system 306. A reduction in the number of communication lines 308 can decrease the potential for cross-talk and can also allow the radial cross-sectional area of device 101, or width, to be minimized, which in turn can allow the introduction of device 101 into smaller regions of the body.

Also, multiplexer 416 can also be used as, or in conjunction with, position adjustable mounting 414 to provide adjustable support for array 106. For instance, in one embodiment, multiplexer 416 is a flexible circuit coupled with array 106. Furthermore, in embodiments where the elements 202 of array 106 are MUTs, multiplexer 416 and array 106 can be monolithically integrated together on a common semiconductor substrate. The integration of multiplexer 416 and array 106 on the same substrate can reduce the size of imaging device 102 and improve the interface performance between array 106 and multiplexer 416.

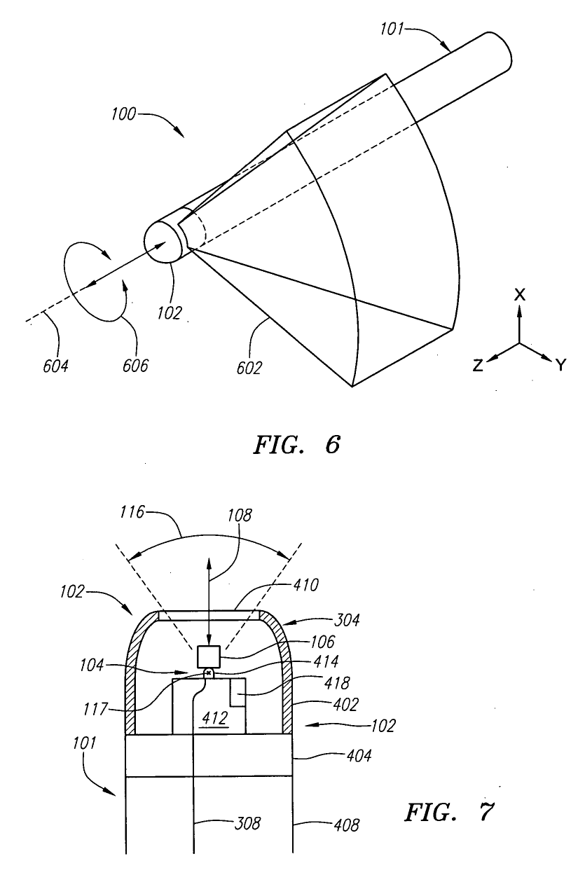

FIG. 6 depicts a perspective view of another example embodiment of imaging system 100 further illustrating the imaging capability of orientation adjustable imaging device 102. In this embodiment, 3D spatial region 602 represents the area that imaging device 102 can image by adjusting the orientation, or pitch, of imaging device 102 in the Z direction and collecting image data from multiple 2D imaging fields 108. Here, imaging device 102 is positioned in a side-looking configuration with respect to medical device 101. Imaging device 102 can also be moved as desired to adjust the overall position of imaging device 102 within the body. For instance, shaft 108 can be moved proximally and distally along central axis 604 and rotated about central axis 604 in direction 606.

FIG. 7 depicts a block diagram of another example embodiment of imaging system 100. Here, imaging device 102 is positioned in a forward-looking configuration with respect to medical device 101. Here, the orientation of array 106 can adjusted across motion range 116 to allow imaging of body tissue located distal to the distal end 304 of medical device 101. One of skill in the art will readily recognize that imaging device can be positioned in any manner within medical device 101 and at any location on medical device 101. In this embodiment, forward-looking array 106 can be an annular array with a symmetric or non-symmetric beam pattern, a non-diffraction array and the like.

In the foregoing specification, the invention has been described with reference to specific embodiments thereof. It will, however, be evident that various modifications and changes may be made thereto without departing from the broader spirit and scope of the invention. For example, each feature of one embodiment can be mixed and matched with other features shown in other embodiments. Features and processes known to those of ordinary skill may similarly be incorporated as desired. Additionally and obviously, features may be added or subtracted as desired. Accordingly, the invention is not to be restricted except in light of the attached claims and their equivalents.

What is claimed is:

1. A medical ultrasound imaging system for three dimensional imaging of a living being, comprising:
an imaging device insertable into a living being and configured to image the interior of the living being, the imaging device comprising:
an ultrasound transducer device having an imaging field; and
an orientation adjustment unit configured to adjust the orientation of the ultrasound transducer device, whereby the imaging field of the ultrasound transducer device changes, the orientation adjustment unit being coupled with the ultrasound transducer device by a flexible circuit.

2. The system of claim 1, wherein the ultrasound transducer device comprises a plurality of transducer elements.

3. The system of claim 1, wherein the ultrasound transducer device is a linear array of a plurality of transducer elements.

4. The system of claim 2, wherein the orientation adjustment unit adjusts the orientation of the ultrasound transducer device by moving the transducer device linearly.

5. The system of claim 2, wherein the orientation adjustment unit adjusts the orientation of the ultrasound transducer device by moving the transducer device in a nonlinear manner.

6. The system of claim 3, wherein the orientation adjustment unit adjusts the orientation of the array by selecting a different transducer element in the array that is permitted to emit acoustic energy.

7. The system of claim 3, wherein the imaging field is located substantially in a first dimension and a second dimension.

8. The system of claim 7, wherein the orientation of the ultrasound transducer device is adjustable about the axis from a first position to a second position, such that the imaging field of the ultrasound transducer device at the first position is separated from the imaging field of the ultrasound transducer device in a second position in a third dimension.

9. The system of claim 8, wherein at least one of the transducer elements is a piezoelectric transducer element.

10. The system of claim 8, wherein at least one of the transducer elements is a capacitive micro-machined ultrasound transducer (CMUT) element.

11. The system of claim 1, wherein the orientation adjustment unit comprises an orientation control unit configured to control the orientation of the ultrasound transducer device.

12. The system of claim 11, wherein the orientation control unit is configured to electrically control the orientation of the ultrasound transducer device.

13. The system of claim 11, wherein the orientation control unit is configured to magnetically control the orientation of the ultrasound transducer device.

14. The system of claim 11, wherein the orientation control unit is configured to mechanically control the orientation of the ultrasound transducer device.

15. The system of claim 11, wherein the orientation adjustment unit further comprises a multiplexer.

16. The system of claim 15, wherein the array is an array of transducer elements, and wherein the multiplexer comprises a first plurality of communication ports, each of the transducer elements being communicatively coupled with a communication port.

17. The system of claim 16, wherein the multiplexer further comprises a second plurality of communication ports and is configured to multiplex signals input to the first plurality of communication ports from the transducer elements onto the second plurality of communication ports.

18. The system of claim 17, wherein the second plurality of communication ports comprises less ports than the first plurality of communication ports.

19. The system of claim 16, wherein the multiplexer further comprises a second plurality of communication ports and is configured to demultiplex signals input to the second plurality of communication ports onto the first plurality of communication ports, wherein the second plurality of communication ports comprises less ports than the first plurality of communication ports.

20. The system of claim 15, wherein the flexible circuit includes the multiplexer.

21. The system of claim 15, wherein each of the transducer elements is a capacitive micro-machined ultrasound transducer (CMUT) element.

22. The system of claim 21, wherein the multiplexer is integrated with the ultrasound transducer device on a common semiconductor substrate.

23. The system of claim 1, wherein the orientation adjustment unit is configured to control the rate of adjustment of the ultrasound transducer device.

24. The system of claim 1, wherein the orientation adjustment unit is configured to determine the orientation of the ultrasound transducer device.

25. The system of claim 2, wherein the ultrasound transducer device is configured to image in an imaging direction, and wherein the imaging direction is at a first angular position in the imaging field.

26. The system of claim 25, wherein the ultrasound transducer device is configured to image in a plurality of different imaging directions, each imaging direction located at a different angular position in the imaging field.

27. The system of claim 26, wherein the ultrasound transducer device is communicatively coupled with an image processing system configured to receive an output signal from each element in the ultrasound transducer device, wherein one or more of the output signals are representative of an echo received in the imaging direction.

28. The system of claim 27, wherein the image processing system is configured to control the imaging direction.

29. The system of claim 28, wherein the image processing system is configured to process the one or more output signals into echo data and store the echo data in an echogenic record.

30. The system of claim 29, wherein one echogenic record is generated for each angular position imaged by the ultrasound transducer device.

31. The system of claim 30, wherein the ultrasound transducer device is configured to image at a first orientation and a second orientation and the image processing system is configured to store echogenic data records generated at the first orientation in a first image data set and echogenic data records generated at the second orientation are stored in a second image data set.

32. The system of claim 31, wherein the image processing system is configured to display the first and second data sets as a three dimensional image.

33. The system of claim 27, wherein the image processing system is configured to generate a three dimensional image of a region imaged by the ultrasound transducer device.

34. The system of claim 1, wherein the ultrasound transducer device is a single transducer element.

35. A method of three dimensional imaging with a medical ultrasound imaging system, comprising:
imaging a first imaging field with an ultrasound imaging device located within a living being;
adjusting the orientation of the ultrasound imaging device with an orientation adjustment unit coupled with the ultrasound imaging device by a flexible circuit; and
imaging a second imaging field with the ultrasound imaging device, the second imaging field being different than the first.

36. The method of claim 35, wherein adjusting the orientation of the ultrasound imaging device comprises pivoting the ultrasound imaging device about an axis.

37. The method of claim 35, wherein the imaging field is located substantially in a first dimension and a second dimension.

38. The method of claim 37, wherein adjusting the orientation of the ultrasound imaging device comprises adjusting the orientation of the imaging device in a third dimension.

39. The method of claim 38, further comprising:
collecting image data from the first and second imaging fields; and
generating a three dimensional image from the collected image data.

40. The method of claim 39, wherein generating a three dimensional image comprises assembling the image data from the first and second imaging fields.

41. The method of claim 35, further comprising controlling the rate of adjustment of the ultrasound imaging device.

42. A medical ultrasound imaging system for three dimensional imaging of a living being, comprising:
an imaging device insertable into-a living being and configured to image the interior of the living being, the imaging device comprising:
an ultrasound transducer device having an imaging field; and
an orientation adjustment unit coupled with the ultrasound transducer device and configured to adjust the orientation of the ultrasound transducer device, the orientation adjustment unit comprising a sensor for sensing the orientation of the ultrasound transducer device.

43. The system of claim 42, wherein the ultrasound transducer device is a linear array of transducer elements.

44. The system of claim 42, wherein the imaging field is located substantially in a first dimension and a second dimension.

45. The system of claim 44, wherein the orientation of the ultrasound transducer device is adjustable about the axis from a first position to a second position, such that the imaging field of the ultrasound transducer device at the first position is separated from the imaging field of the ultrasound transducer device in a second position in a third dimension.

46. The system of claim 42, wherein the orientation adjustment unit comprises an orientation control unit configured to control the orientation of the ultrasound transducer device.

47. The system of claim 46, wherein the orientation adjustment unit further comprises an adjustable mounting, wherein the ultrasound transducer device is adjustably mounted thereon.

48. The system of claim 42, wherein the orientation adjustment unit is configured to control the rate of adjustment of the ultrasound transducer device.

49. The system of claim 42, wherein the orientation adjustment unit is configured to determine the orientation of the ultrasound transducer device with the sensor.

50. The system of claim 42, wherein the ultrasound transducer device is configured to image with an ultrasound beam, the beam direction being adjustable.

51. The system of claim 42, wherein the ultrasound transducer device is configured to image in an imaging direction, and wherein the imaging direction is at a first angular position in the imaging field.

52. The system of claim 51, wherein the ultrasound transducer device is configured to image in a plurality of different imaging directions, each imaging direction located at a different angular position in the imaging field.

53. The system of claim 52, wherein the ultrasound transducer device is communicatively coupled with an image processing system configured to receive an output signal from each element in the ultrasound transducer device, wherein one or more of the output signals are representative of an echo received in the imaging direction and wherein the image processing system is configured to process the one or more output signals into echo data and store the echo data in an echogenic record.

54. The system of claim 53, wherein the image processing system is configured to control the imaging direction.

55. The system of claim 54, wherein one echogenic record is generated for each angular position imaged by the ultrasound transducer device.

56. The system of claim 55, wherein the ultrasound transducer device is configured to image at a first orientation and a second orientation and wherein the image processing system is configured to store echogenic data records generated at the first orientation in a first image data set and echogenic data records generated at the second orientation are stored in a second image data set.

57. The system of claim 56, wherein the image processing system is configured to display the first and second data sets as a three dimensional image.

58. The system of claim 53, wherein the image processing system is configured to generate a three dimensional image of a region imaged by the ultrasound transducer device.
